(19) United States
(12) Patent Application Publication
(10) Pub. No.: US 2016/0030233 A1
(43) Pub. Date: Feb. 4, 2016

(54) APPARATUSES AND METHODS FOR COOLING A SURFACE

(71) Applicant: EMPIRE TECHNOLOGY DEVELOPMENT LLC, Wilmington, DE (US)

(72) Inventors: Benjamin William MILLAR, Rosebery (AU); George Charles PEPPOU, Hornsby Heights (AU); Michael Keoni MANION, Seattle, WA (US); Maggie Chai Cin NG, Blacktown (AU)

(21) Appl. No.: 14/449,571

(22) Filed: Aug. 1, 2014

(51) Int. Cl. A61F 7/00; F25B 21/02

(52) U.S. Cl. CPC A61F 7/007; F25B 21/02; A61F 2007/0004; A61F 2007/0075; A61F 2007/0087

(57) ABSTRACT

Methods and apparatuses for providing localized cooling of a surface are described. Localized cooling devices may include a thermal element configured to generate a temperature gradient in a cooling interface in contact with the surface. Temperature gradient may operate to facilitate conductive heat transfer from the surface, through the cooling interface, and out into the ambient environment. The thermal elements may include thermoelectric cooling elements (TECs), such as Peltier cooling elements. In some embodiments, the localized cooling devices may be configured to treat swelling associated with periorbital edema by providing convenient, efficient, and continuous cooling of the affected area.

### BACKGROUND

Localized swelling of human tissue may be caused by various factors, such as inflammation and/or the buildup of fluid in the tissue. A common condition caused by such localized swelling is periorbital edema, which generally involves the swelling of tissue (or “puffiness”) in the orbital region surrounding the eye. Periorbital edema may in some cases be a sign of a serious medical condition, such as hypothyroidism, an eye infection (for example, periorbital cellulitis), Chagas disease, and trichinosis. However, most people experience periorbital edema as a temporary cosmetic issue due to allergies, normal aging, excess salt consumption, lack of sleep, or the buildup of fluid in the tissues that sometimes occurs during sleep.

Conventional non-surgical techniques for treating periorbital edema include applying cold compresses and/or astringents, such as a sliced cucumber, a wet teabag, or calamine lotion, to the swollen area. However, such techniques often provide limited success and are difficult to apply on a consistent basis. In addition, such conventional techniques are not portable and generally require application in a person's home. Accordingly, it would be beneficial to treat periorbital edema using a device capable of providing consistent, effective treatment in a manner that is simple to perform, portable, and discreet.

### SUMMARY

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

In an embodiment, an apparatus may be configured to provide localized cooling of a surface. The apparatus may include at least one cooling interface configured to contact the surface and at least one solid-state thermoelectric element thermally coupled to the at least one cooling interface, the at least one solid-state thermoelectric element being configured to facilitate a conductive transfer of heat from the surface through the at least one cooling interface, thereby decreasing a temperature of the surface.

In an embodiment, a method of cooling a surface may include providing at least one cooling interface configured to contact the surface. At least one solid-state thermoelectric element may be thermally coupled to the at least one cooling interface to facilitate a conductive transfer of heat from the surface through the at least one cooling interface. Power may be provided to the at least one solid-state thermoelectric element to facilitate the conductive transfer of heat. The at least one cooling interface may be contacted with the surface, thereby decreasing a temperature of the surface.

In an embodiment, a method of manufacturing an apparatus configured to provide localized cooling of a surface may include providing at least one cooling interface configured to contact the surface, providing at least one solid-state thermoelectric element, thermally coupling the at least one solid-state thermoelectric element to the at least one cooling interface, and configuring the at least one solid-state thermoelectric element to facilitate a conductive transfer of heat from the surface through the at least one cooling interface, thereby decreasing a temperature of the surface.

In an embodiment, a method of cooling human skin located in a periorbital region of a human face to reduce swelling thereof due to periorbital edema may include providing at least one cooling interface configured to contact the human skin, the at least one cooling interface being shaped to correspond to the periorbital region of a human face, thermally coupling at least one solid-state thermoelectric element to the at least one cooling interface to facilitate a conductive transfer of heat from the human skin through the at least one cooling interface, providing power to the at least one solid-state thermoelectric element to facilitate the conductive transfer of heat, and contacting the at least one cooling interface with the human skin, thereby decreasing a temperature of the human skin.

### BRIEF DESCRIPTION OF THE DRAWINGS

FIG. 1 depicts an illustrative localized cooling device according to some embodiments.

FIGS. 2A and 2B depict an illustrative hand-held localized cooling device configured to treat periorbital edema according to some embodiments.

FIG. 3 depicts an illustrative method of treating periorbital edema using a localized cooling device according to some embodiments.

FIG. 4 depicts a flow diagram for an illustrative method of localized cooling of a surface according to some embodiments.

### DETAILED DESCRIPTION

The terminology used in the description is for the purpose of describing the particular versions or embodiments only, and is not intended to limit the scope.

The described technology generally relates to apparatuses and methods for providing localized cooling of a surface, such as the surface of an object or human skin. In general, the localized cooling may occur by conducting heat from the surface to the apparatus (the “localized cooling device”) and out into the environment. The localized cooling device may include an interface (a “cooling interface”) configured to contact the surface to be cooled. A thermal element may be thermally coupled to the interface to facilitate the transfer of heat from the surface to the cooling interface. For example, the transfer of heat may occur through conductive heat transfer. In some embodiments, the thermal element may include a Peltier cooling element. In some embodiments, the thermal element may facilitate the transfer of heat from the surface to the cooling interface by producing a temperature gradient in the at least one cooling interface. The thermal element may be configured to facilitate the cooling of the surface responsive to receiving power from a power supply. In some embodiments, the power supply may be activated and/or controlled by a user of the localized cooling device. In some embodiments, the localized cooling device may be configured as a hand-held device suitable for personal use, such as to reduce the swelling of skin for cosmetic reasons. For example, the localized cooling device may be configured to decrease the temperature of tissue (for instance, skin, blood vessels, and other tissue below the skin) in a periorbital region of a human face to reduce swelling (or “puffiness”) in the periorbital region (for instance, due to periorbital edema).

In this manner, a user may be able to treat conditions involving swollen or inflamed tissue, such as the periorbital region of the human face due to periorbital edema, using a convenient and portable hand-held device that may be controlled by the user. Use of the described technology can result in a reduction or elimination of swelling of human tissue in a more effective and efficient manner relative to treatment of the same or similar conditions without the described methods and devices. The degree of swelling of human tissue can generally be reduced by any amount. For example, the degree of swelling can be reduced by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, and in an ideal situation, about 100% reduction (complete elimination of swelling). The reduction in degree of swelling can be determined by measurement before and after use of the device.

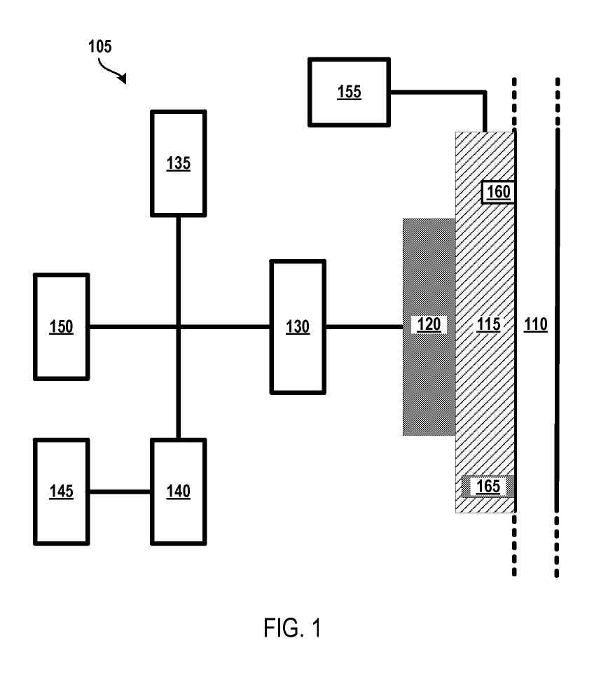

FIG. 1 depicts an illustrative localized cooling device according to some embodiments. As shown in FIG. 1, a localized cooling device 105 may be configured to cool a surface 110. The surface 110 may generally include the surface of any object, material, or the like capable of being cooled according to some embodiments. Non-limiting examples of a surface 110 may include a metal, a ceramic, a glass material, a polymer material, a fiber-based material, a fluid, a gel, the skin of a living being, such as human skin, the surface of an electronic device, or the like.

The localized cooling device may include a cooling interface 115 configured to contact the surface 110. The cooling interface 115 may be formed from various materials, such as materials configured to facilitate heat transfer. Non-limiting examples of cooling interface 115 materials may include a metal, a ceramic, stainless steel, aluminum, anodized aluminum, elastomers, silicone, graphite, neoprene, alloys thereof, particles thereof, and combinations thereof. In some embodiments, the cooling interface 115 and/or materials thereof may be modified to increase the thermal conductivity of the cooling interface. For example, materials of the cooling interface 115, such as an elastomer layer or a neoprene layer, may be modified with high thermal conductivity silicone rubber with graphite or aluminum particles.

The cooling interface 115 may have various thicknesses. In general, the thickness of the cooling interface 115 may include any thickness capable of facilitating heat transfer according to some embodiments. In some embodiments, the cooling interface 115 may be sized and/or shaped to facilitate heat transfer and/or to correspond to the shape and/or contour of the surface 110. For example, the cooling interface 115 may have a size, shape, and/or contour configured to maximize contact between the cooling interface 115 and the surface 110. In some embodiments, the cooling interface 115 may be sized and/or shaped to conform to a particular cooling application. For instance, in an embodiment in which the cooling interface 115 is configured to cool the periorbital region of the eye, the cooling interface may have a shape and size configured to conform to the periorbital region of the eye, such as a shape having a diameter of about 50 millimeters to about 70 millimeters.

In some embodiments, the cooling interface 115 may be bendable, flexible, soft, pliable, or otherwise have a degree of elasticity. In such embodiments, the cooling surface 115, may flex and/or bend to conform or substantially conform to the shape of the surface 110, such as the periorbital region of a human face. In this manner, the cooling surface 115 may be configured to provide a more custom fit for surfaces than would otherwise be achieved with more rigid and non-flexible cooling surfaces. In some embodiments, the cooling interface 115 may include a flexible portion and a non-flexible (or comparatively more rigid portion). For instance, the cooling interface 115 may include a thin flexible layer over a rigid base having high thermal conductivity. In a non-limiting example, the cooling interface 115 may be formed from a silicone layer (flexible layer) arranged over an aluminum base (base having high thermal conductivity). In some embodiments in which the flexible layer includes a low conductivity material (in comparison with a high conductivity material such as aluminum), the thickness of the flexible layer may be minimized so as not to obstruct the thermal conductivity of the base having high thermal conductivity. In some embodiments, the flexible layer may have a thickness of about 1 millimeter, about 2 millimeters, about 3 millimeters, about 5 millimeters, or values or ranges between any two of these values (including endpoints).

A thermal element 120 may be thermally coupled to the cooling interface 115. The thermal element 120 may include any element capable of facilitating the transfer of heat and, therefore, the cooling of the surface 110. In some embodiments, the transfer of heat may occur through conductive heat transfer. In some embodiments, the thermal element 120 and the cooling interface 115 may be separate components. For example, the thermal element 120 may be physically attached to or otherwise thermally coupled to the cooling interface 115. In some embodiments, the cooling interface 115 may be configured as a layer or other component of the thermal element 120 that is a part of or is integral to the thermal element. For example, the cooling interface 115 may include an outer portion of the thermal element 120 configured to contact the surface 110. In some embodiments, a thin film of thermally conductive grease (for example, silicone grease) may be arranged on the cooling interface 115 to eliminated gaps in thermal coupling.

In some embodiments, the thermal element 120 may include a thermo-chemical element configured to cool an object or provide heat transfer through a chemical reaction. In some embodiments, the thermal element 120 may include a thermoelectric element (a thermoelectric cooler (TEC)) configured to use electricity to generate a heat gradient or heat flux for transferring heat from a first location (for instance, the surface 110) to a second location (for instance, the environment external to the thermal element 120). In some embodiments, the thermal element 120 may include a solid-state thermoelectric element. In some embodiments, the thermal element 120 may include a thermoelectric element configured to operate using the Peltier effect (a “Peltier cooling element”) to transfer heat from a first side of the thermal element (for instance, a side facing the surface 110) to a second side of the thermal element (for instance, a side opposite the side facing the surface). In some embodiments, the thermal element 120 may include one or more a solid-state Peltier cooling devices as known to those having ordinary skill in the art. In some embodiments, the thermal element 120 may include a thin film TEC. In some embodiments, the localized cooling device 105 may include one thermal element 120. In some embodiments, the localized cooling device 105 may include a plurality of thermal elements 120.

The thermal element 120 may use various levels of electrical power to operate according to some embodiments. In an embodiment in which the thermal element 120 is or uses one or more Peltier coolers, the thermal element may require about 2.5 Watts to about 12.5 Watts per about 100 millimeters² of surface area (for example, cooling surface area) of the cooling surface 115, for a total power requirement of about 10 Watts to about 50 Watts. The actual power required to cool a surface according to some embodiments may be a fraction of this power requirement, depending, for example, on specific cooler efficiency levels.

The thermal element 120 may have various sizes according to some embodiments. In some embodiments, the thermal element may have an area of about 100 millimeters², about 150 millimeters², about 200 millimeters², about 300 millimeters², about 500 millimeters², about 1000 millimeters², and any value or range between any two values (including endpoints). In some embodiments, the thermal element 120 may include a plurality of thermal elements, such as a plurality of Peltier cooling elements. In some embodiments, the thermal element 120 may include 2 thermal elements, 3 thermal elements, 4 thermal elements, 5 thermal elements, 10 thermal elements, 20 thermal elements, or any value or range between any two values (including endpoints).

The thermal element 120 may be powered (or “activated”) through a power supply 130 controlled through a switch 135. The power supply 130 may include any type of power supply capable of powering the thermal element 120 according to some embodiments. In some embodiments, the power supply 130 may include an electrical power supply, such as a battery power supply. The switch 135 may include a pressure switch and/or any other type of switch capable of controlling power to the thermal element 120. In some embodiments, the thermal element 120 may be electrically coupled to various safety switches (not shown) configured to disconnect the power supply 130 from the thermal element, or otherwise power-off the thermal element, responsive to various conditions. For example, safety switches may be configured to detect over-current and/or over-temperature conditions and to disconnect power to the thermal element 120 responsive to detection of unsafe conditions relating thereto.

The localized cooling device 105 may include a timing element 140 configured to provide time information associated with the use of the localized cooling device and components thereof. In some embodiments, the timing element 140 may be configured to determine a length of time that the thermal element 120 has been activated. The localized cooling device 105 may include an interface contact element 165 configured to determine when the cooling interface 115 is in contact with the surface 110. For example, the interface contact element 165 may be configured to detect the pressure produced by pressing the cooling interface 115 against the surface 110. In some embodiments, the timing element 140 may be configured to determine a length of time that the cooling interface 115 has been in contact with the surface 110. In some embodiments, the timing element 140 may be configured to determine a length of time that the thermal element 120 has been activated and the cooling interface 115 has been in contact with the surface 110. In some embodiments, the timing element 140 may be configured to count down a predetermined time duration. In some embodiments, the predetermined time duration may be specified by a user.

The timing element 140 may be configured to initiate an event responsive to expiration of a predetermined time period, such as a user-specified time duration, a length of time associated with activation of the thermal element 120, a length of time associated with contact between the cooling interface 115 and the surface 110, and/or a length of time associated with activation of the thermal element 120 and contact between the cooling interface 115 and the surface 110. In some embodiments, the event may include, without limitation, powering off the thermal element 120, initiating an audible alarm, and/or initiating a visible alarm. In some embodiments, the localized cooling device 105 may include an alarm element 145 configured to facilitate an audible alarm and/or a visible alarm. In some embodiments, the alarm element 145 may include a speaker and/or a light.

In some embodiments, the localized cooling device 105 may include a temperature sensor (not shown) configured to detect the temperature of the surface 110 in contact with or immediately adjacent to the cooling interface 115. In some embodiments, the localized cooling device 105 may be configured to provide a visual readout of the temperature detected by the temperature sensor. In some embodiments, the localized cooling device 105 may be configured to generate an event responsive to the temperature falling above/below a predetermined threshold. In some embodiments, the event may include an audible alarm and/or a visible alarm. In some embodiments, the predetermined threshold may be specified by a user. In some embodiments, the predetermined threshold may be specified within the localized cooling device 105, for example in hardware and/or software. In some embodiments, the predetermined threshold may be selected in order to prevent discomfort, damage, or the like to the skin of a user.

The localized cooling device 105 may include a control element 150 operatively coupled to the thermal element 120 and/or the power supply 130. The control element 150 may be configured to control the thermal element 120, such as the rate of heat transfer facilitated by the thermal element. For example, the control element 150 may be configured to control the temperature of the thermal element 120 (for instance, the level of “cold” of the thermal element). In some embodiments, the control element 150 may be configured to control the transfer of heat by controlling an amount of power being provided to the thermal element 120. In some embodiments, the control element 150 may be or may include a potentiometer.

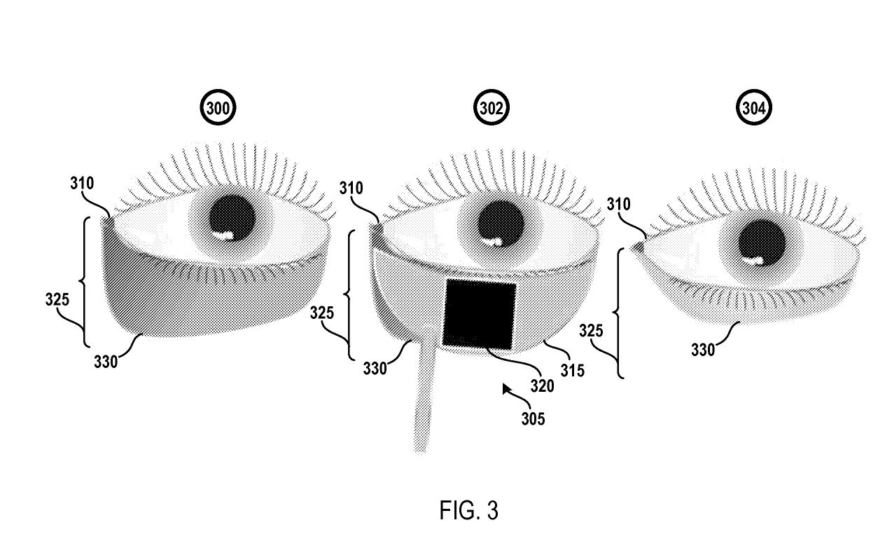

In some embodiments, the cooling interface 115 may be configured to dispense a fluid onto the surface 110. A fluid reservoir 155 storing the fluid may be in fluid communication with the cooling interface 115. The fluid reservoir 155 may in some cases be replaceable or refillable. The cooling interface 115 may receive the fluid from the fluid reservoir 155 and may include one or more discharge openings 160 configured to dispense the fluid onto the surface 110. The cooling interface 115 may receive the fluid from the fluid reservoir 155 responsive to activation of an actuator (not shown; see FIG. 3). In some embodiments, the fluid may include a cream, a liquid, a gel, or a combination thereof. In some embodiments, the fluid may include an evaporative liquid configured to evaporate at atmospheric temperature and pressure conditions. The evaporative liquid may provide a cooling sensation when evaporating from the surface 110 after being dispensed thereon. In some embodiments, the fluid may include a moisturizing cream, a dermatological medicine, a fluid configured to block ultraviolet rays from reaching the skin, an anti-inflammatory material, an alcohol, or some combination thereof. In some embodiments, the fluid may include at least one therapeutically active ingredient. In some embodiments, the therapeutically active ingredient may include at least one dermatological material. In some embodiments, the at least one dermatological material may include cortisone, calamine, turmeric, or a combination thereof.

In an embodiment configured for treating periorbital edema, the fluid may include an astringent and/or a facial cooling gel known to those having ordinary skill in the art. The heat transfer coefficient of the boundary between human skin and the cooling interface 115 may be increased by the presence of such an astringent and/or facial cooling gel because such substances may reduce or eliminate low conductivity air gaps.

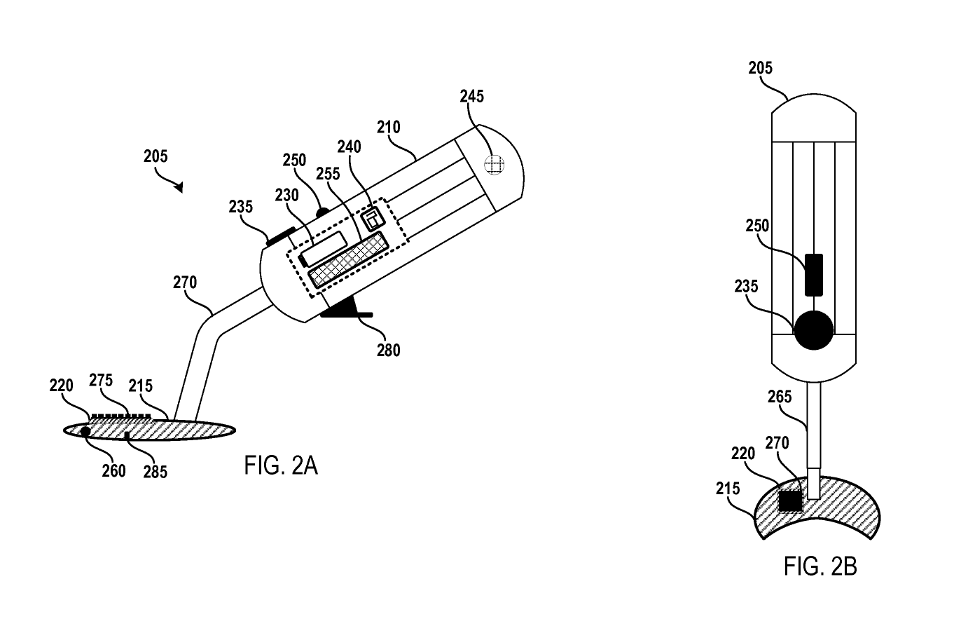

FIGS. 2A and 2B depict an illustrative hand-held localized cooling device configured to treat periorbital edema according to some embodiments. In particular, FIG. 2A depicts a side view of the illustrative hand-held localized cooling device and FIG. 2B depicts a top-down view of the illustrative hand-held localized cooling device.

Periorbital edema may be caused by the swelling of tissue surrounding the eye, including skin and blood vessels in the periorbital regions of the human face. Although periorbital edema may be caused by and/or indicative of a serious medical condition, it is generally considered to be a cosmetic condition that may negatively affect the physical appearance and/or the perceived age of an affected person. In most non-medical cases, the swelling may be rapidly reduced by cooling the affected tissue and adjacent areas. As shown in FIGS. 2A and 2B, some embodiments provide a hand-held localized cooling device 205 configured to cool the periorbital area of a human face to treat tissue swelling associated with periorbital edema.

The localized cooling device 205 may include a handle 210 configured, for example, to be held by the hand of a user in a manner similar to holding a flashlight handle. The user may actuate a switch 235 to activate a thermal element 220. The thermal element 220 may include a solid-state thermoelectric element, such as a Peltier cooling element as known to those having ordinary skill in the art. In some embodiments, actuation of the switch 235 may cause a power supply 230 to provide power to the thermal element 220. In some embodiments, the localized cooling device 205 may be configured such that a user may efficiently actuate the switch 235 using a thumb of the hand holding the handle. In some embodiments, detection of contact between a cooling interface 215 and a surface by a contact sensor 285 may cause the power supply 230 to provide power to the thermal element 220. In some embodiments, the power supply 230 may include a battery. In some embodiments, the battery may include a rechargeable battery. When power is delivered to the thermal element 220 heat may be drawn from a surface contacting the cooling interface 215, such as human skin in the periorbital region of a human face. The heat may be drawn from the surface, through the cooling interface 215 and the thermal element 220, through cooling fins 275 in thermal communication with the thermal elements, and into the environment. In this manner, human skin in contact with the cooling interface 215 may be cooled by the localized cooling device 205.

In some embodiments, the cooling interface 215 may have a size, shape, and/or contour configured to correspond to the size, shape, and/or contour of the periorbital region of the human face. For example, the cooling interface 215 may have a concave or substantially concave shape configured to envelope the convex or substantially convex shape of the periorbital region of the human face, particularly when there is swollen tissue in the periorbital region.

In some embodiments, the localized cooling device 205 may be configured to decrease the temperature of human skin by a moderate degree, such as about 2° C. to about 10° C. In some embodiments, the localized cooling device 205 may be configured to decrease the temperature of human skin by about 2° C., about 4° C., about 5° C., about 6° C., about 8° C., about 10° C., about 20° C., or about any value or range between any two of these values (including endpoints). In some embodiments, the localized cooling device 205 may be configured to decrease the temperature of human skin by about 2° C. to about 10° C. after about 30 seconds of contact with the human skin. In some embodiments, the localized cooling device 205 may be configured to decrease the temperature of human skin by about 2° C. to about 10° C. after contacting the human skin for about 10 seconds, about 20 seconds, about 30 seconds, about 40 seconds, about 50 seconds, or about 60 seconds. In some embodiments, the localized cooling device 205 may be configured to provide a level of cooling substantially similar to placing an ice cube on the swollen tissue; however, capable of being carried out indefinitely and without the melting and handling challenges associated with using an actual ice cube.

A shaft 270 may connect the handle 210 to the cooling element 215. The shaft 270 may include and/or operate as a conduit for connections between various elements including, without limitation, the power supply 230 and the thermal element 220, the fluid reservoir 255 and a discharge opening 260, the timing element 240 and the thermal element 220 and/or the contact detector 285, and/or any other connections within the localized cooling device 205. In some embodiments, the timing element 240 may be operatively coupled to the switch 235, the battery 230, and/or an alarm element 245 in order to initiate an event according to some embodiments. In some embodiments, the fluid reservoir 255 may be operatively coupled to an actuator 280 that may cause fluid to be dispensed from the discharge opening 260. In some embodiments, the actuator 280 may be a trigger-style actuator configured to be actuated by a finger of the hand holding the handle 210.

A control element 250 may be arranged on the handle 210 to allow a user to control the rate of cooling (for instance, by controlling the temperature of the thermal element 220). A non-limiting example of a control element 250 may include a switch configured to provide “low,” “medium,” and “high” settings. In some embodiments, the rate of cooling may be controlled by control elements 250 configured to operate based on user preferences. In some embodiments, the user may set a predetermined cooling duration such that the thermal element 220 may be de-activated after expiration of the predetermined cooling duration.

As shown in FIG. 2A, certain components of the localized cooling device may be located internally, such as in the handle 210 (as indicted by the cut-out lines). For instance, the power supply 230, the fluid reservoir 255, the timer 240, the alarm element 245, and/or any electrical and/or fluid connections associated therewith, may be located at least partially within or fully within the handle 210.

FIG. 3 depicts an illustrative method of treating periorbital edema using a localized cooling device according to some embodiments. At step 300, a portion of the periorbital region 325 of a human eye 310 may include swollen tissue 330 having a certain initial area. In step 302, a cooling interface 315 operatively coupled to an activated thermal element 320 of a localized cooling device 305 may be contacted with the swollen tissue 330 for a particular duration. As shown in step 304, the swollen tissue 330 of the periorbital region 325 has been substantially reduced and/or eliminated. Accordingly, application of the localized cooling device 305 may operate to efficiently and effectively reduce the swelling of tissue 330 in the periorbital region 325 of the human face.

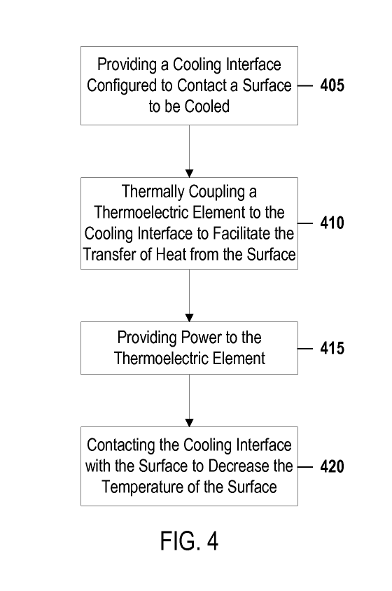

FIG. 4 depicts a flow diagram for an illustrative method of localized cooling of a surface according to some embodiments. A cooling interface may be provided 405 that is configured to contact a surface to be cooled. In some embodiments, the cooling interface may be shaped, sized, or otherwise configured to correspond with the shape, size, and/or any other characteristic of the surface to be cooled. In this manner, the cooling interface may be configured to maximize contact with the surface. A thermoelectric element may be thermally coupled 410 to the cooling interface to facilitate the transfer of heat from the surface. In some embodiments, the thermoelectric element may include a solid-state thermoelectric element configured to transfer heat using the Peltier effect as known to those having ordinary skill in the art. Power may be provided 415 to the thermoelectric element to initiate the transfer of heat via the thermoelectric element. The cooling interface may be contacted 420 with the surface to decrease the temperature of the surface. The thermoelectric element may operate to facilitate the transfer of heat of a surface in contact with the cooling interface from the surface, through the cooling interface and thermoelectric element, and into the ambient atmosphere. In this manner, the thermoelectric element may operate to decrease the temperature of the surface.

### EXAMPLES

### Example 1

### Multi-Surface Localized Cooling Device

A cooling device is configured to provide localized cooling for various types of surfaces, including metal surfaces and polymer-based surfaces. The cooling device includes a handle having a battery disposed therein to provide power to an array of 5 solid-state Peltier cooling elements. One rechargeable CR123A battery is used. The array of Peltier cooling elements are arranged on a first surface of a ceramic cooling interface having a substantially circular shape and an area of about 20 centimeters². Each of the Peltier cooling elements has a copper electrical conductor and an aluminum fin on its cold side. The working power of each Peltier cooling element is about 9.5 Watts and the maximum temperature difference between the hot side and the cold side (the “delta T”) is about 70° C.

The localized cooling device may be used to decrease the temperature of various surfaces within a manufacturing facility that become heated during the manufacturing process and that need to be cooled rapidly to allow workers to continue working. A power switch located on the outside of the handle is configured to provide power to the array of Peltier cooling elements responsive to actuation by a user. The second side of the cooling interface, opposite the first side, may be contacted with a surface to be cooled. A temperature sensor detects the temperature of the surface in contact with the cooling interface and the localized cooling device provides an audible signal when the temperature of the surface is at a safe temperature for working, namely, 35° C. or below.

### Example 2

### Periorbital Edema Treatment Device

A periorbital edema treatment device (the “treatment device”) may include a cooling interface formed from polished stainless steel. The cooling side of the cooling interface may have a size and a slightly concave shape to correspond with the shape of the periorbital region of a human face in order to maximize the contact area of the cooling interface and the periorbital region. The non-cooling side of the cooling interface may have 10 thin-film TEC thermal elements arranged thereon. Each thin-film TEC has an area of about 1 centimeter and a delta T of about 70° C.

The thermal elements are activated by pressing the cooling interface against the skin of a user. The cooling side of the cooling interface includes a pressure switch configured to provide power to the thermal elements responsive to determining when the cooling interface is being pressed against a surface. The treatment device includes a potentiometer configured to manage the amount of power and, therefore, amount of cooling being provided by the thermal elements. The potentiometer may be controlled by a user via a control switch configured to provide selections for “low,” “medium,” and “high” levels of cooling. A timer is started responsive to activation of the thermal elements. The timer generates an audible signal (a “beep”) after a predetermined amount of time of 60 seconds has expired.

A volume of skin cream is arranged within a reservoir in the handle of the treatment device. The skin cream includes the therapeutically active ingredient cortisone. The skin cream may be dispensed from the reservoir and onto the skin through openings in the cooling side of the cooling interface. The skin cream also includes an astringent configured to improve the heat transfer coefficient of the treatment device. The reservoir is configured such that it is removable and replaceable by the user, for example, once the skin cream contents have been fully dispensed.

The treatment device at the “medium” cooling level may reduce the temperature of skin in contact with the cooling side of the cooling interface by about 5° C. in about 30 seconds. The treatment device can substantially eliminate swelling of tissue in the periorbital region in about 60 seconds.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to”). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example), the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, or the like. As a non-limiting example, each range discussed herein can be readily broken down into a lower third, a middle third, and an upper third. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. An apparatus configured to provide localized cooling of a surface, the apparatus comprising:
at least one cooling interface configured to contact the surface; and
at least one solid-state thermoelectric element thermally coupled to the at least one cooling interface, the at least one solid-state thermoelectric element being configured to facilitate a conductive transfer of heat from the surface through the at least one cooling interface, thereby decreasing a temperature of the surface.

2. The apparatus of claim 1, wherein the at least one solid-state thermoelectric element comprises at least one Peltier cooling element.

3. The apparatus of claim 1, wherein the at least one solid-state thermoelectric element facilitates the conductive transfer of heat from the surface through the at least one cooling interface by producing a temperature gradient in the at least one cooling interface.

4. The apparatus of claim 1, wherein the at least one cooling interface is shaped to correspond to the periorbital region of a human face.

5. The apparatus of claim 1, wherein the surface comprises human skin and the apparatus is configured to provide localized cooling of the human skin to reduce swelling thereof.

6. The apparatus of claim 5, wherein the apparatus is configured to provide localized cooling of human skin in a periorbital region of a human face to reduce swelling due to periorbital edema.

7. The apparatus of claim 5, wherein the apparatus is configured to decrease the temperature of the human skin by about 2° C. to about 10° C.

8. The apparatus of claim 5, wherein the apparatus is configured to decrease the temperature of the human skin by about 2° C. to about 10° C. after about 30 seconds of contact between the at least one interface and the human skin.

9. The apparatus of claim 5, further comprising at least one fluid dispenser configured to dispense at least one fluid onto the human skin.

10. The apparatus of claim 9, wherein the at least one fluid comprises at least one of the following therapeutically active ingredients: cortisone, calamine and turmeric.

11. The apparatus of claim 1, wherein the at least one cooling interface comprises at least one of a metal, a metal alloy, and a combination thereof.

12. The apparatus of claim 1, further comprising a pressure switch configured to activate the at least one solid-state thermoelectric element.

13. The apparatus of claim 1, further comprising at least one control element operatively coupled to the at least one solid-state thermoelectric element, the at least one control element being configured to control a rate of the conductive transfer of heat.

14. The apparatus of claim 1, further comprising a timing element configured to generate an event responsive to expiration of a predetermined time period, wherein the event comprises at least one of powering off the at least one solid-state thermoelectric element, initiating an audible signal, and initiating a visual signal.

15. A method of cooling a surface, the method comprising:
providing at least one cooling interface configured to contact the surface;
thermally coupling at least one solid-state thermoelectric element to the at least one cooling interface to facilitate a conductive transfer of heat from the surface through the at least one cooling interface;
providing power to the at least one solid-state thermoelectric element to facilitate the conductive transfer of heat; and
contacting the at least one cooling interface with the surface, thereby decreasing a temperature of the surface.

16. The method of claim 15, wherein the at least one solid-state thermoelectric element comprises at least one Peltier cooling element.

17. The method of claim 15, wherein contacting the at least one cooling interface with the surface comprises contacting the at least one cooling interface to human skin to reduce swelling thereof.

18. The method of claim 15, wherein contacting the at least one cooling interface to human skin comprises contacting the at least one cooling interface to human skin in a periorbital region of a human face.

19. The method of claim 15, further comprising controlling a rate of the conductive transfer of heat using at least one control element operatively coupled to the at least one solid-state thermoelectric element.

20. The method of claim 15, further comprising timing a duration of the conductive transfer of heat and generating an event responsive to the duration exceeding a predetermined time period, wherein the event comprises at least one of powering off the at least one solid-state thermoelectric element, initiating an audible signal, and initiating a visual signal.

21. A method of cooling human skin located in a periorbital region of a human face to reduce swelling thereof due to periorbital edema, the method comprising:
providing at least one cooling interface configured to contact the human skin, the at least one cooling interface being shaped to correspond to the periorbital region of a human face;
thermally coupling at least one solid-state thermoelectric element to the at least one cooling interface to facilitate a conductive transfer of heat from the human skin through the at least one cooling interface;
providing power to the at least one solid-state thermoelectric element to facilitate the conductive transfer of heat; and
contacting the at least one cooling interface with the human skin, thereby decreasing a temperature of the human skin.

22. The method of claim 21, wherein the at least one solid-state thermoelectric element comprises at least one Peltier cooling element.

23. The method of claim 21, wherein the temperature of the human skin is decreased by about 2° C. to about 10° C.

24. The method of claim 21, further comprising dispensing at least one fluid onto the human skin using at least one fluid dispenser.

25. The method of claim 24, wherein the at least one fluid comprises at least one therapeutically active ingredient configured to treat swelling of human skin.
